(19) United States
(12) Patent Application Publication
(10) Pub. No.: US 2011/0172460 A1
(43) Pub. Date: Jul. 14, 2011

(54) PROCESS FOR THE MANUFACTURE OF RACEMIC 2-ARYL-PROPIONIC ACID

(75) Inventors: Stephen John Martin, Northumberland (GB); Scott Dale Makin, Curtin (AU)

(73) Assignee: AESICA PHARMACEUTICALS LIMITED, Cramlington, Northumberland (GB)

(21) Appl. No.: 13/001,893

(22) PCT Filed: Jun. 30, 2009
(86) PCT No.: PCT/GB09/01619

§ 371 Date: Mar. 28, 2011

(30) Foreign Application Priority Data
Jun. 30, 2008 (GB) 0811851.5

(51) Int. Cl. C07C 51/347; C07C 57/30; C07C 59/52; C07C 57/38; C07C 59/84

(52) U.S. Cl. 562/401; 562/496; 562/466; 562/492; 562/460

(57) ABSTRACT

There is described a process for the manufacture of a racemic 2-aryl propionic acid compound, or a pharmaceutically acceptable salt thereof, which comprises reacting the S- or R-enantiomer of the corresponding 2-aryl propionic acid compound with a base.

### FIELD OF THE INVENTION

The present invention provides a novel process for the manufacture of racemic 2-aryl propionic acid compounds, and pharmaceutically acceptable salts thereof, such as flurbiprofen, with a reduced methyl ester content and a reduced enantiomeric excess from mixtures containing the 2-aryl propionic acid compound, such as flurbiprofen, enriched in either the S or the R enantiomer.

The invention also provides a novel process for the manufacture of S- and/or R-forms of the 2-aryl propionic acids, and pharmaceutically acceptable salts thereof, such as flurbiprofen, by resolution of the racemic 2-aryl propionic acids, such as flurbiprofen, described herein.

### BACKGROUND OF THE INVENTION

A number of known pharmaceutically active agents which are therapeutically useful as non-steroidal anti-inflammatory drugs (NSAID) and are used to treat, inter alia, inflammation and pain, for example, caused by arthritis, comprise a 2-aryl propionic acid moiety I;

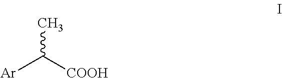

Such 2-aryl propionic acid NSAIDs include, but shall not be limited to, ibuprofen, flurbiprofen, ketoprofen, naproxen, etc., and pharmaceutically acceptable salts thereof.

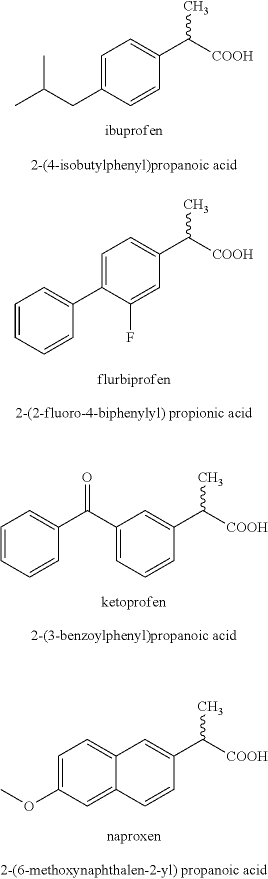

By way of illustration, chemically, flurbiprofen is 2-(2-fluoro-4-biphenylyl)propionic acid and is described in U.S. Pat. No. 3,755,427. NSAIDs, such as flurbiprofen, are usually supplied as a racemate. However, recently there has been renewed interest in the separate enantiomers of flurbiprofen, i.e. S-flurbiprofen and R-flurbiprofen.

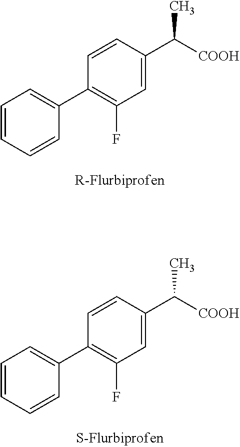

Flurbiprofen is a potent inhibitor of cyclooxygenase (both COX-1 and COX-2) in humans and it is understood that the inhibitory effect lies predominantly in the S-enantiomer.

Flurbiprofen is generally produced in the form of a racemic compound. It is known that from the racemic compound, flurbiprofen having a high optical purity can be produced by an optical resolution method using, for example, an optically active amine compound, such as α-phenylethylamine, as an optical resolution agent, as is described in U.S. Pat. No. 5,599,969.

In addition, whether dealing with racemic, S- or R-2-aryl propionic acid, there is also a need to make the synthetic process as efficient as possible.

In the existing manufacturing process for either the S- or the R-2-aryl propionic acid compound, and pharmaceutically acceptable salts thereof, unwanted enantiomer will generally be racemised by first converting it into the methyl ester, as is described for ibuprofen and flurbiprofen in U.S. Pat. No. 5,599,969, allowing subsequent resolution of the racemate and enabling a higher yield of the desired enantiomer to be achieved.

However, the rate at which undesired enantiomer can be reconverted to the racemic 2-aryl propionic acid compound in order to be re-used in the process can act as a bottleneck and can hold back the capacity of the process. For example, one batch may take several days to complete.

Thus, for example, the current commercial process for the manufacture of flurbiprofen comprises resolution of flurbiprofen in a toluene methanol mixture. The toluene/methanol mother liquors from the resolution steps are combined and the methanol is removed, for example, by distillation. Phenylethylamine (PEA) is removed by being washed out. The flurbiprofen left behind will predominantly comprise the undesired enantiomer, which is then esterified, for example, by refluxing in methanol with catalytic sulphuric acid. The substantially enantiomeric flurbiprofen methyl ester is then saponified, during which it also undergoes racemisation. The product from this racemisation reaction can then be used in the resolution process so as to increase the yield of the desired R- or S-enantiomer of flurbiprofen. This process is represented in the schematic below for racemisation of S-flurbiprofen, although it will be understood by the person skilled in the art that an equivalent process may be used for racemisation of R-flurbiprofen:

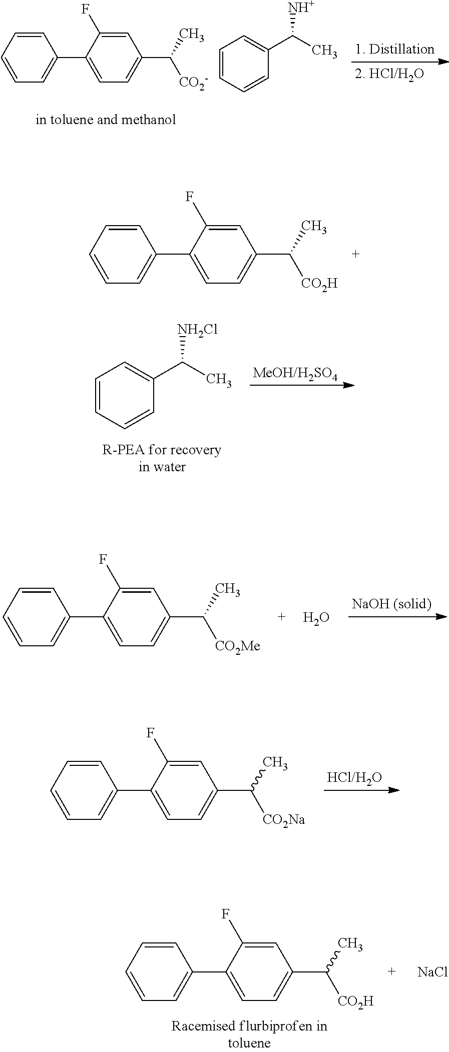

However, the process described above is known to produce certain undesirable impurities in the final product, for example, methyl (2-(2-fluoro-4-biphenylyl)) propionate (I); 1-phenylethyl-(2-2(fluoro-4-biphenylyl)) propionamide (II):

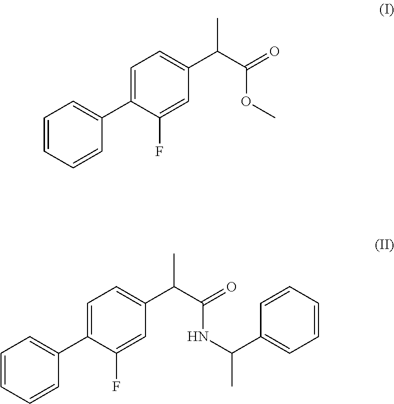

It is understood by the person skilled in the art that similar analogous impurities may be produced in process used for the manufacture of other 2-aryl propionic acid compounds.

However, we have now found a novel process in the manufacture of racemic-aryl propionic acid compounds.

### SUMMARY OF THE INVENTION

The novel process of manufacturing racemic flurbiprofen provides a method of preparing an enantiomeric S- and/or R-form of a 2-aryl propionic acid compound. The process provides greater efficiency than existing processes via an improved racemisation step. The process is also advantageous in that, inter alia, it produces the racemic 2-aryl propionic acid compound with a reduced methyl ester content and/or a reduced enantiomeric excess.

Therefore, according to a first aspect of the invention we provide a process for the manufacture of a racemic 2-aryl propionic acid compound, or a pharmaceutically acceptable salt thereof, which comprises reacting the S- or R-enantiomer of the corresponding 2-aryl propionic acid compound with a base.

It will be understood that this base reaction is intended to convert the undesired enantiomer to the racemic 2-aryl propionic acid compound and therefore the reaction will generally be carried out on the “waste” product of a resolution step. Whilst this base reaction will generally be carried out on a single enantiomer, due to the nature of the “waste” material, some of the desired enantiomer may also be present.

It will be understood by the person skilled in the art that by the term “2-aryl propionic acid compound” we generally mean any derivative which comprises a chiral centre in a 2-phenyl propionic acid moiety, in which the phenyl group may be fused or substituted, etc.

However, particular examples of 2-aryl propionic acid compounds which may be mentioned are 2-(4-isobutylphenyl)propanoic acid (ibuprofen), 2-(2-fluoro-4-biphenylyl)propionic acid (flurbiprofen), 2-(3-benzoylphenyl)propanoic acid (ketoprofen), and 2-(6-methoxynaphthalen-2-yl)propanoic acid (naproxen), and pharmaceutically acceptable salts thereof.

The base may vary depending upon, inter alia, the 2-aryl propionic acid compound, but it is generally preferably an anionic base, for example, comprising hydroxide ions. Desirably the base is not an amine base. Thus, the anionic base may comprise one or more alkali metal salts, such as sodium or potassium. Thus, a most preferred base is sodium hydroxide.

The amount of base present may vary and is preferably from 2.5 to 10 equivalents based on the quantity of the S- and R-enantiomer of the 2-aryl propionic acid compound which is present, preferably 4 to 7 equivalents, more preferably 5 to 6 equivalents.

An important aspect of the present invention is the use of a solvent mixture in the process of the invention. Although the solvent mixture may be varied according to the desired 2-aryl propionic acid compound, preferably, the solvent comprises a mixture of a water miscible solvent, such as an alcohol, e.g. an alkyl C1 to C10 alcohol, preferably an alkyl C1 to 6 alcohol, and a water immiscible solvent, such as a hydrocarbon solvent.

By the term “alkyl” we mean a fully saturated branched or unbranched hydrocarbon moiety, i.e. primary, secondary or tertiary alkyl or, where appropriate, cycloalkyl or alkyl substituted by cycloalkyl. Where not otherwise identified, the alkyl may comprise 1 to 10 carbon atoms, preferably 1 to 6 carbon atoms or 1 to 4 carbon atoms. Examples of such alkyl groups include, but shall not be limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl, n-decyl and the like.

Thus, the alkyl C1 to C6 alcohol may be, for example, methanol, ethanol, n-propanol or iso-propanol.

The water immiscible solvent may comprise, for example, long chain alcohols, hexane, cyclohexane, chloroform, and tetrachloroethylene, ethyl acetate, isopropyl acetate, and methyl isobutyl ketone, petroleum solvents or aromatic solvents, such as toluene.

The solvent mixture may be multi-component, i.e. may comprise more than two components, however, preferably the mixture is a two component mixture. An especially preferred hydrocarbon solvent is toluene. Thus, an especially preferred solvent mixture is toluene and an alkyl C1 to C6 alcohol.

Surprisingly we have found that, even though an object of the invention is to avoid the necessity of production of the methyl ester of the undesired enantiomer of the 2-aryl propionic acid compound n, the most desirable alkyl alcohol co-solvent is generally methanol. Methanol as a co-solvent is especially desirable when the 2-aryl propionic acid compound is flurbiprofen or alternatively ibuprofen.

Therefore, we further provide a process as hereinbefore described wherein the process includes the use of a solvent mixture, wherein at least one component of the mixture is methanol. Thus, the mixture may comprise methanol and a water immiscible solvent, such as a hydrocarbon solvent as hereinbefore defined. The most preferred solvent mixture is methanol and toluene.

The ratio of the water immiscible solvent to the water miscible solvent may vary depending, inter alia, upon the nature of the solvents. Thus, for example ratio of water immiscible: water miscible may be from 1:1 to 10:1 v/v, preferably from 2:1 to 8:1 and especially from 3:1 to 5:1, e.g. 4:1 v/v. When the solvent mixture comprises a methanol/toluene mixture the ratio of toluene: methanol may vary, but it may be from 1:1 to 10:1 v/v, preferably from 2:1 to 8:1 and especially from 3:1 to 5:1, e.g. 4:1 v/v.

The temperature at which the racemisation reaction is carried out may vary, but is preferably at the reflux temperature of the solvent mixture, which is in the range 60 to 65° C. for a methanol/toluene mixture as hereinbefore described, since a methanol-toluene azeotrope boils at a lower temperature than methanol alone.

The reaction time may also vary depending upon, inter alia, the 2-aryl propionic acid compound, but may be from 3 to 10 hours, preferably 5 to 8 hours, for example 6 hours

The process of the first aspect of the invention may be schematically represented as follows:

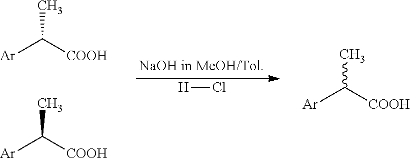

Thus, by way of example the schematic process of the invention for flurbiprofen may be represented as follows:

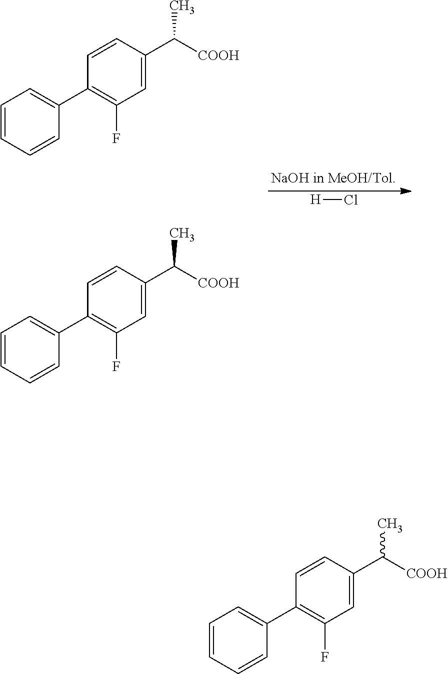

The racemate produced by the process of the invention is useful, inter alia, as an intermediate in the manufacture of the desirable enantiomers, e.g. in the case of flurbiprofen, S-flurbiprofen and R-flurbiprofen; or in the case of ibuprofen, S-ibuprofen and R-ibuprofen; etc. Furthermore, the racemate of the 2-aryl propionic acid compound produced according to this aspect of the invention may have reduced methyl ester content and/or a reduced enantiomeric excess.

Thus, the invention also provides a racemic 2-aryl propionic acid compound, as hereinbefore described, or a pharmaceutically acceptable salt thereof, as hereinbefore described wherein it has an enantiomeric excess of substantially zero, i.e. 0.04% w/w or less. Thus, the racemic 2-aryl propionic acid compound according to this aspect of the invention may be, for example, racemic ibuprofen, racemic flurbiprofen, racemic ketoprofen or racemic naproxen, and pharmaceutically acceptable salts thereof, wherein it has an enantiomeric excess of substantially zero, i.e. 0.04% w/w or less.

We further provide the racemic 2-aryl propionic acid compound, or a pharmaceutically acceptable salt thereof, as hereinbefore described wherein the amount of the corresponding methyl propionate ester impurity is less than 2.5% w/w.

Preferably, the amount of the corresponding methyl propionate ester impurity is less than 2.5% w/w, more preferably less than 2%, more preferably less than 1.5% w/w and especially less than 1.0% w/w.

Thus, for example, in the case of flurbiprofen, the invention provides racemic flurbiprofen, or a pharmaceutically acceptable salt thereof, wherein the amount of methyl (2-(2-fluoro-4-biphenylyl)) propionate impurity is less than 2.5% w/w.

According to a further aspect of the invention we provide a process for the manufacture of an enantiomer of a 2-aryl propionic acid compound, as hereinbefore described, or a pharmaceutically acceptable salt thereof, which comprises the resolution of the corresponding racemic 2-aryl propionic acid compound, or a salt thereof, produced according to the invention as hereinbefore described. Thus, according to this aspect of the invention the process for the manufacture of an enantiomer of, for example, ibuprofen, flurbiprofen, ketoprofen or naproxen, and pharmaceutically acceptable salts thereof.

Thus, for flurbiprofen we provide a process for the manufacture of an enantiomer of flurbiprofen, i.e. S-2-(2-fluoro-4-biphenylyl)propionic acid, or a salt thereof, or R-2-(2-fluoro-4-biphenylyl)propionic acid, or a salt thereof, which comprises the resolution of racemic flurbiprofen, or a salt thereof, produced according to the invention as hereinbefore described.

More particularly, we provide a process for the manufacture of an enantiomer of a 2-aryl propionic acid compound, or a pharmaceutically acceptable salt thereof, which comprises the steps of:

(i) resolution of the racemic 2-aryl propionic acid compound, or a pharmaceutically acceptable salt thereof, by reaction with a resolving agent;
(ii) separation of the desired enantiomer from the undesired enantiomer;
(iii) racemisation of the undesired enantiomer which comprises reacting the undesired enantiomer with a base to produce the racemic 2-aryl propionic acid compound;
(iv) resolution of the racemic 2-aryl propionic acid compound, or a pharmaceutically acceptable salt thereof, of step (iii).

In a preferred aspect of the invention the process for the manufacture of an enantiomer as hereinbefore described comprises the manufacture of an enantiomer of flurbiprofen, i.e. S-2-(2-fluoro-4-biphenylyl)propionic acid, or a salt thereof, or R-2-(2-fluoro-4-biphenylyl)propionic acid, or a salt thereof, which comprises the steps of: (i) resolution of racemic flurbiprofen, or a salt thereof, by reaction with a resolving agent;

(ii) separation of the desired enantiomer from the undesired enantiomer;
(iii) racemisation of the undesired enantiomer which comprises reacting the undesired enantiomer with a base to produce racemic flurbiprofen;
(iv) resolution of racemic flurbiprofen, or a salt thereof, of step (iii).

In this aspect of the invention any conventionally known resolving agent may be considered in the process of the invention. Thus, the resolving agent may vary depending upon, inter alia, the 2-aryl propionic acid compound. However, a preferred resolving agent is a resolving base, such as an amine base, for example, 1-phenylethylamine (PEA). For the manufacture of S-enantiomer of the 2-aryl propionic acid compound from the racemic compound, the resolving agent is preferably S-1-phenylethylamine. Similarly, for the manufacture of R-enantiomer of the 2-aryl propionic acid compound from the racemic flurbiprofen, the resolving agent is preferably R-1-phenylethylamine.

In one aspect of the present invention relates to racemic or enantiomeric 2-(4-isobutylphenyl)propanoic acid (ibuprofen), and pharmaceutically acceptable salts thereof. In another aspect the present invention relates to racemic or enantiomeric 2-(2-fluoro-4-biphenylyl)propionic acid (flurbiprofen), and pharmaceutically acceptable salts thereof. In another aspect the present invention relates to racemic or enantiomeric 2-(3-benzoylphenyl)propanoic acid (ketoprofen), and pharmaceutically acceptable salts thereof. In another aspect the present invention relates to racemic or enantiomeric 2-(6-methoxynaphthalen-2-yl)propanoic acid (naproxen), and pharmaceutically acceptable salts thereof.

Examples of suitable pharmaceutically acceptable salts the API include, but are not limited to, aluminium, calcium, lithium, magnesium, potassium, sodium and zinc. In addition, organic salts may also be used including, but not limited to salts of lysine, N,N′-dibenzylethylenediamine, chloroprocaine, choline, diethanolamine, ethylenediamine, meglumine (N-methylglucamine), procaine and tromethamine.

The invention will now be described by way of example only. The following examples are intended to illustrate the invention and are not to be construed as being limitations thereon. Temperatures are given in degrees centigrade. If not mentioned otherwise, all evaporations are performed under reduced pressure, preferably between about 15 mm Hg and 100 mm Hg (=20-133 mbar). The structure of final products, intermediates and starting materials is confirmed by standard analytical methods, e.g., microanalysis and spectroscopic characteristics, e.g., MS, IR, NMR, etc. Abbreviations used are those conventional in the art. All starting materials, building blocks, reagents, acids, bases, dehydrating agents, solvents, and catalysts utilized to synthesis the compounds of the present invention are either commercially available or can be produced by organic synthesis methods known to one of ordinary skill in the art.

### EXAMPLE 1

### Flurbiprofen

### Example 1.1

### Resolution Procedure

Racemic flurbiprofen (3.0 kg) was charged to a 20 L jacketed glass reactor. Methanol (2.0 L) and toluene (8.0 L) were added. The mixture was heated to dissolve the solid. S-1-Phenylethylamine (0.76 kg) was dissolved in toluene (1.87 L) and the solution was added to the 20 L reactor with stirring at 60° C. over about 30 minutes. The mixture was cooled gradually to 0 to 5° C. to induce crystallisation. The crystals were filtered off, washed with toluene (3 L) and dried in a vacuum oven at 55° C. to form crude S-flurbiprofen/S-1-phenylethylamine salt (1.4 kg).

Crude S-flurbiprofen/S-1-phenylethylamine salt (1.0 kg) was charged to a 20 L jacketed glass reactor. Toluene (11.4 L) and methanol (2.7 L) were added and the mixture was stirred and heated to 60° C. to dissolve the solid. The solution was cooled gradually to 0 to 5° C. to induce crystallisation. The crystals were filtered off, washed with toluene (3 L) and dried in a vacuum oven at 55° C. to form pure S-flurbiprofen/S-1-phenylethylamine salt (0.8 kg).

Pure S-flurbiprofen/S-1-phenylethylamine salt (2.2 kg) was charged to a 20 L jacketed glass reactor. Toluene (7.5 L) was added with stirring. Water (1.9 L) and concentrated hydrochloric acid (0.9 L) were added and the mixture was stirred at 60° C. The lower aqueous layer was separated off and the upper organic layer was retained. The hydrochloric acid wash was repeated, then the toluene solution was washed with water. Additional toluene (1.2 L) was added and then toluene (3.5 L) was distilled off to ensure the solution was free from water. The toluene solution was cooled gradually to −10° C. to induce crystallisation. The crystals were filtered off, washed with heptane (1.9 L) and dried in a vacuum oven at 40° C. to form pure S-flurbiprofen (1.1 kg).

### Racemisation Procedure

Toluene/methanol mother liquors from the filtration of crude S-flurbiprofen/S-1-phenylethylamine salt in the resolution procedure (2 L, containing an estimated 370 g of flurbiprofen) were charged into a 3 L 3 necked round bottomed flask and methanol was distilled out at atmospheric pressure (volume removed approximately 250 ml). The batch was then cooled to around 60° C. and washed twice with hydrochloric acid (80 ml concentrated hydrochloric acid in 330 ml of water), and then twice with water (330 ml). Toluene was charged (160 ml) followed by methanol (388 ml) and caustic soda solution (800 ml of 28% w/w solution, 5 molar equivalents). The mixture was heated to reflux for about 6 hours. Solvent was then removed at atmospheric pressure until the vapour temperature reached approximately 85° C. The mixture was cooled to around 60° C. and concentrated hydrochloric acid was charged at about 60 to 70° C. until the pH of the mixture was 1 or less. The layers were allowed to separate and the bottom aqueous layer removed. The organic layer was washed with water (400 ml) and then azeotroped to dryness using a Dean and Stark trap. A solution of racemic flurbiprofen in toluene remained.

### Example 1.2

### Variation of Solvent Quantities

S-Flurbiprofen (20 g) was charged to a 500 ml jacketed glass reactor. Toluene (200 ml) was added and the solution was heated to 60° C. Sodium hydroxide solution (46 ml of 28% w/w solution, 5.1 molar equivalents) and methanol (70 ml) were added. The mixture was heated to reflux for 4 hours. Solvent was removed by distillation until the distillate temperature reached 100° C. and the volume removed was replaced with toluene. The reaction mixture was neutralised by addition of hydrochloric acid (60 ml of 36% w/w) at 60° C. and the lower aqueous layer was separated off. The organic layer containing the flurbiprofen was analysed and found to have an enantiomeric excess of 5.6%.

The above procedure was repeated at half the scale using different solvent proportions. S-flurbiprofen (10 g) was mixed with toluene (100 ml) and methanol (20 ml). Sodium hydroxide solution (25 ml of 28% w/w solution, 5.6 molar equivalents) was added and the mixture heated to reflux for 3 hours. After distillation of solvent and neutralisation by addition of hydrochloric acid (35 ml of 36% w/w solution), the organic layer was found to contain flurbiprofen with enantiomeric excess of 3.4%.

### Example 1.3

### Variation of Amount of Sodium Hydroxide

Toluene/methanol mother liquors from the filtration of crude R-flurbiprofen/R-1-phenylethylamine salt in the resolution steps (250 ml, containing an estimated 47 g of flurbiprofen) were charged into a 500 ml jacketed glass reactor and methanol was distilled out at atmospheric pressure. The mixture was then cooled to around 60° C. and washed twice with hydrochloric acid (40 ml concentrated hydrochloric acid in 100 ml of water), and then twice with water (100 ml). Methanol was charged (50 ml) followed by caustic soda solution (see table). The mixture was heated to reflux (see table). Solvent was then removed at atmospheric pressure until the vapour temperature reached approximately 85° C. The mixture was cooled to around 60° C. and concentrated hydrochloric acid was charged at about 60 to 70° C. until the pH of the mixture was 1 or less. The layers were allowed to separate and the bottom aqueous layer removed. The organic layer was washed with water and then azeotroped to dryness using a Dean and Stark trap. The remaining solution of racemic flurbiprofen in toluene was analysed for enantiomeric purity.

| Sodium hydroxide | | |
|---|---|---|
| quantity (molar | | |
| equivalents) | Reflux time | Enantiomeric excess |
| 3.0 | 6 h | 11.9 |
| 4.4 | 3.5 h | 0.8 |
| 5.8 | 4 h | 0.2 |

### Example 1.4

### Large Scale Trial

Toluene/methanol mother liquors from the filtration of crude R-Flurbiprofen/R-1-Phenylethylamine salt in the resolution steps (11.5 L, containing an estimated 1.7 kg of flurbiprofen enriched in the S-enantiomer) were charged into a 20 L jacketed glass reactor and methanol was distilled out at atmospheric pressure. The mixture was then cooled to around 60° C. and washed twice with hydrochloric acid (0.6 L concentrated hydrochloric acid in 1.8 L of water), and then twice with water (1.9 L). Additional toluene was charged (1.0 L) followed by methanol (2.5 L) and caustic soda solution (4.5 L of 28% w/w solution, 6.1 molar equivalents). The mixture was heated to reflux for 6 hours. Solvent was then removed at atmospheric pressure until the vapour temperature reached approximately 85° C. The mixture was cooled to around 60° C. and concentrated hydrochloric acid (4.5 L of 36% w/w solution) and water (2 L) was charged at about 60 to 70° C. until the pH of the mixture was 1 or less. Further toluene (3 L) was added and the layers were allowed to separate and the bottom aqueous layer removed. The organic layer was washed with water (3.6 L) and then azeotroped to dryness using a Dean and Stark trap. The remaining solution of racemic flurbiprofen in toluene was analysed giving the results shown below. Typical results obtained from batches carried out using the prior art method for the racemisation as described in U.S. Pat. No. 5,599,969 are shown in the Table for comparison.

| | | |
|---|---|---|
| | | Typical result from |
| Parameter | Result (%) | prior art process (%) |

Enantiomeric Purity

| | | |
|---|---|---|
| R-flurbiprofen content | 50.02 | 48.5 to 50.0 |
| S-Flurbiprofen content | 49.98 | 50.0 to 51.5 |
| Enantiomeric excess | 0.04 | 0 to 3 |

Related substances by HPLC

| | | |
|---|---|---|
| 2-(4-biphenylyl) | 0.25 | 0.2 to 0.3 |
| propionic acid | | |
| Methyl (2-(2-fluoro-4- | 0.6 | 1 to 5 |
| biphenylyl)) propionate | | |
| 1-phenylethyl-(2- | 1.7 | 1 to 5 |
| 2(fluoro-4-biphenylyl)) | | |
| propionamide | | |

### Example 1.5

### Synthesis of R-Flurbiprofen/R-1-Phenylethylamine Salt from Racemised Flurbiprofen

Toluene/methanol mother liquors from the filtration of crude R-flurbiprofen/R-1-phenylethylamine salt in the resolution step (1.25 L, containing an estimated 230 g of flurbiprofen enriched in the S-enantiomer) were charged into a 3 L 3 necked round bottomed glass reactor and methanol (150 ml) was distilled out at atmospheric pressure. The mixture was then cooled to around 60° C. and washed twice with hydrochloric acid (50 ml concentrated hydrochloric acid in 200 ml of water), and then twice with water (250 ml). Additional toluene was charged (100 ml) followed by methanol (180 ml), caustic soda solution (250 ml of 48% w/w solution, 4.7 molar equivalents) and water (250 ml). The mixture was heated to reflux for 6 hours. Solvent was then removed at atmospheric pressure until the vapour temperature reached approximately 85° C. The mixture was cooled to around 60° C. and further toluene (380 ml) was added. Hydrochloric acid (500 ml of 36% w/w solution) was charged at about 60 to 70° C. until the pH of the mixture was 1 or less and the layers were allowed to separate and the bottom aqueous layer removed. The organic layer was washed with water (250 ml) and then azeotroped to dryness using a Dean and Stark trap. The remaining solution of racemic flurbiprofen in toluene was analysed and found to have an enantiomeric excess of 1.1%. A portion of the resulting solution (160 g) was evaporated to dryness to determine its flurbiprofen content (22 g) then redissolved in toluene (50 g) and methanol (11 g). The solution was heated to 60° C. and a solution of R-1-phenylethylamine (6.5 g) in toluene (14 g) was added slowly. The mixture was cooled slowly to 0° C. and the crystals were filtered off, washed with toluene (25 ml) and dried under vacuum at 60° C. Analysis showed an enantiomeric excess of 77%, typical for crude R-flurbiprofen/R-1-phenylethylamine salt. Levels of methyl (2-(2-fluoro-4-biphenylyl)) propionate and 1-phenylethyl-(2-2(fluoro-4-biphenylyl)) propionamide were only 0.1%.

### EXAMPLE 2

### Ibuprofen

### Example 2.1

### Resolution Procedure

Racemic ibuprofen (530 g) is dissolved in toluene (1335 ml) and methanol (900 ml). The mixture is heated to dissolve the solid. S-1-Phenylethylamine (247 g) is dissolved in toluene (200 ml) and the solution is added with stirring at 60° C. over about 3 hours while the temperature is maintained at about 65-70° C. The mixture is cooled gradually to 0 to 5° C. to induce crystallisation and stirred at this temperature for 1 hour. The crystals are filtered off, washed with toluene (600 ml) and dried in a Vacuum oven at 55° C. to form crude S-ibuprofen/S-1-phenylethylamine salt (635 g).

Crude S-ibuprofen/S-1-phenylethylamine salt (635 g) is stirred with toluene (1930 ml) and methanol (800 ml) and the mixture is heated to 60° C. to dissolve the solid. The solution is cooled gradually to 0 to 5° C. to induce crystallisation. The crystals are filtered off and dried in a vacuum oven at 55° C. to form pure S-ibuprofen/5-1-phenylethylamine salt (510 g). This recrystallisation of the S-ibuprofen/S-1-phenylethylamine salt may be repeated if necessary to upgrade the enantiomeric purity if required.

Pure S-ibuprofen/S-1-phenylethylamine salt (485 g) is mixed with toluene (1700 ml) with stirring. Water (300 ml) and concentrated hydrochloric acid (170 g) are added and the mixture is stirred at 60° C. The lower aqueous layer is separated off and the upper organic layer is retained. The hydrochloric acid wash is repeated, then the toluene solution is washed with water. Water (370 ml) and 47% sodium hydroxide solution (118 g) are added and the solution is heated to 60° C. and allowed to settle. The lower aqueous layer is separated and the upper toluene layer is washed with water. The aqueous phases are combined and heptane (420 ml) is added. Hydrochloric acid (130 g) is added and the mixture is heated to 60° C., stirred and settled. The organic layer is separated off and washed with water. The solution is cooled to −10° C. to induce crystallisation and the crystals are separated off by filtration, washed with heptane and dried under vacuum to yield (S)-ibuprofen (280 g) at an enantiomeric purity of over 99%.

### Example 2.2

### Racemisation Procedure

Toluene/methanol mother liquors from the filtration of crude S-ibuprofen/S-1-phenylethylamine salt in the resolution procedure (2400 ml, containing an estimated 130 g of ibuprofen) is charged into a 3 L 3 necked round bottomed flask and methanol and toluene are distilled out at atmospheric pressure (volume removed approximately 1400 ml). The batch is then cooled to around 60° C. and washed twice with hydrochloric acid (20 ml concentrated hydrochloric acid in 200 ml of water), and then twice with water (200 ml). Toluene is charged (80 ml) followed by methanol (200 ml) and caustic soda solution (450 g of 28% w/w solution, 5 molar equivalents). The mixture is heated to reflux for about 6 hours. Solvent is then removed at atmospheric pressure until the vapour temperature reaches approximately 85° C. The mixture is cooled to around 60° C. and concentrated hydrochloric acid is charged at about 60 to 70° C. until the pH of the mixture is 1 or less. The layers are allowed to separate and the bottom aqueous layer removed. The organic layer is washed with water (200 ml) and then azeotroped to dryness using a Dean and Stark trap. A solution of racemic ibuprofen in toluene remains.

1. A process for the manufacture of a racemic 2-aryl propionic acid compound, or a pharmaceutically acceptable salt thereof, which comprises reacting the S- or R-enantiomer of the corresponding 2-aryl propionic acid compound with a base.

2. A process according to claim 1 wherein the 2-aryl propionic acid compound is selected from the group consisting of 2-(4-isobutylphenyl)propanoic acid (ibuprofen), 2-(2-fluoro-4-biphenylyl)propionic acid (flurbiprofen), 2-(3-benzoylphenyl)propanoic acid (ketoprofen), and 2-(6-methoxynaphthalen-2-yl)propanoic acid (naproxen), and pharmaceutically acceptable salts thereof.

3. A process according to claim 1 wherein the base comprises one or more alkali metal salts.

4. (canceled)

5. A process according to claim 1 wherein the base comprises a hydroxide.

6. (canceled)

7. A process according to claim 1 wherein the amount of base present is from 2.5 to 10 equivalents relative to the 2-aryl propionic acid compound present.

8. A process according to claim 1 wherein the reaction is carried out in a solvent mixture comprising an alkyl C1 to C10 alcohol and a water immiscible solvent.

9. (canceled)

10. A process according to claim 8 wherein the water immiscible solvent is toluene.

11. A process according to claim 10 wherein the ratio of toluene: methanol is from 2:1 to 8:1 v/v.

12. A racemic 2-aryl propionic acid compound, or a pharmaceutically acceptable salt thereof, manufactured by a process according to claim 1.

13. A racemic 2-aryl propionic acid compound, or a pharmaceutically acceptable salt thereof, according to claim 12 wherein the racemate has an enantiomeric excess of substantially zero.

14. A racemic 2-aryl propionic acid compound, or a pharmaceutically acceptable salt thereof, according to claim 12 wherein the racemate has a corresponding methyl propionate ester impurity content of less than 2.5% w/w.

15. A racemic 2-aryl propionic acid compound, or a pharmaceutically acceptable salt thereof, according to claim 12 wherein the 2-aryl propionic acid compound is selected from the group consisting of 2-(4-isobutylphenyl)propanoic acid (ibuprofen), 2-(2-fluoro-4-biphenylyl)propionic acid (flurbiprofen), 2-(3-benzoylphenyl)propanoic acid (ketoprofen), and 2-(6-methoxynaphthalen-2-yl)propanoic acid (naproxen), and pharmaceutically acceptable salts thereof.

16. A process for the manufacture of an enantiomer of a 2-aryl propionic acid compound, or a pharmaceutically acceptable salt thereof, which comprises the resolution of a corresponding racemic 2-aryl propionic acid compound, or a salt thereof, according to claim 12.

17. A process for the manufacture of an enantiomer of a 2-aryl propionic acid compound, or a pharmaceutically acceptable salt thereof, which comprises the steps of:
(i) resolution of the racemic 2-aryl propionic acid compound, or a pharmaceutically acceptable salt thereof, by reaction with a resolving agent;
(ii) separation of the desired enantiomer from the undesired enantiomer;
(iii) racemisation of the undesired enantiomer which comprises reacting the undesired enantiomer with a base to produce the racemic 2-aryl propionic acid compound;
(iv) resolution of the racemic 2-aryl propionic acid compound, or a pharmaceutically acceptable salt thereof, of step (iii).

18. A process for the manufacture of S-2-(2-fluoro-4-biphenylyl)propionic acid, or a salt thereof, or R-2-(2-fluoro-4-biphenylyl)propionic acid, or a salt thereof, which comprises the steps of:
(i) resolution of racemic 2-(2-fluoro-4-biphenylyl)propionic acid, or a salt thereof, by reaction with a resolving agent;
(ii) separation of the desired enantiomer from the undesired enantiomer;
(iii) racemisation of the undesired enantiomer which comprises reacting the undesired enantiomer, or a pharmaceutically acceptable salt thereof, with a base;
(iv) resolution of racemic 2-(2-fluoro-4-biphenylyl)propionic acid, or a salt thereof, of step (iii).

19. A process according to claim 18 wherein the resolving agent is an amine base.

20. A process according to claim 19 wherein the amine base is 1-phenylethylamine.

21. A process according to claim 20 wherein the resolving agent is S-1-phenylethylamine or R-1-phenylethylamine.

22. (canceled)

23. (canceled)

24. The S-enantiomer of a 2-aryl propionic acid compound, or a pharmaceutically acceptable salt thereof, manufactured by a process according to claim 18.

25. The R-enantiomer of a 2-aryl propionic acid compound, or a pharmaceutically acceptable salt thereof, manufactured by a process according to claim 18.

26. (canceled)

27. (canceled)

28. The S-enantiomer according to claim 24, wherein the 2-aryl propionic acid compound, or a pharmaceutically acceptable salt thereof, is S-2-(2-fluoro-4-biphenylyl)propionic acid.

29. The R-enantiomer according to claim 25, wherein the 2-aryl propionic acid compound, or a pharmaceutically acceptable salt thereof, is R-2-(2-fluoro-4-biphenylyl)propionic acid.

30. A process according to claim 17 wherein the resolving agent is an amine base.

31. A process according to claim 30 wherein the amine base is 1-phenylethylamine.

32. A process according to claim 31 wherein the resolving agent is S-1-phenylethylamine or R-1-phenylethylamine.

33. The S-enantiomer of a 2-aryl propionic acid compound, or a pharmaceutically acceptable salt thereof, manufactured by a process according to claim 17.

34. The R-enantiomer of a 2-aryl propionic acid compound, or a pharmaceutically acceptable salt thereof, manufactured by a process according to claim 17.
